(19) United States
(12) Patent Application Publication
(10) Pub. No.: US 2018/0105781 A1
(43) Pub. Date: Apr. 19, 2018

(54) METHOD FOR THREE DIMENSIONAL PRINTING ARTIFICIAL SKIN

(71) Applicant: 3D GLOBAL BIOTECH INC., New Taipei City (TW)

(72) Inventors: Keng-Liang OU, New Taipei City (TW); Chih-Hua Yu, New Taipei City (TW); Yu-Hao Chan, New Taipei City (TW); Wei-Jen Cheng, New Taipei City (TW)

(21) Appl. No.: 15/783,368

(22) Filed: Oct. 13, 2017

(30) Foreign Application Priority Data
Oct. 14, 2016 (TW) 105133251

(51) Int. Cl. C12M 3/00; C09D 11/108; A61F 2/10; A61L 27/60; A61L 27/24

(52) U.S. Cl. CPC C12M 21/08; C09D 11/108; C12N 2502/094; A61L 27/60; A61L 27/24; A61F 2/105

(57) ABSTRACT

The present invention discloses a method for three dimensional printing artificial skin. The method comprises the following steps of: printing a dermis layer by a hydrogel combined with an autologous dermal cell and; printing an epidermis layer on the dermis layer by a hydrogel combined with an autologous kerationocytes cell; implanting a plurality of progenitor cells for sweat glands and hair follicles through the epidermis layer into the dermis layer to form an artificial skin. Because all used materials are from the autologous skin, and the 3D printing technology is used to implant the progenitor cells for sweat glands and hair follicles to form the artificial skin which has sweat glands and hair follicles, the present invention can get an artificial skin with a complete skin structure.

### CROSS REFERENCE TO RELATED APPLICATIONS

This application claims priority to Taiwan Application No. 105133251, filed Oct. 14, 2016, entitled “A METHOD FOR THREE DIMENSIONAL PRINTING ARTIFICIAL SKIN”, reference of which is hereby incorporated in its entirety.

### BACKGROUND OF THE INVENTION

### Field of the Invention

The present invention relates to a method of forming an artificial skin, more particularly, to a method of 3D printing an artificial skin with sweat glands and hair follicles.

### Description of the Prior

The skin is the body's largest organizational structure. The skin is not only regulating the body temperature and preventing the body of water loss, but also serving as the front line to defense the external pathogens and the bacterial infections. When the skin has a large area of damage, the human body may be dehydrated and infected by bacteria, which can result in death.

Traditional methods of skin damage that cannot be healed naturally comprise the following: to get a portion of the skin from the other parts of the patient and then to lay it on the wound; or to get the skin from other people or mammals (e.g. pigs) and then to lay it on the wound. However, the former method cannot get a lot of skin because the other parts of the body can be easily infected by the bacteria in doing so. The latter method has an advantage of mass-production, but the biocompatibility problems may cause death easily. In order to solve the above problems, the artificial skin technology is developed.

In the prior art, an animal skin containing an epidermal layer and a dermal layer from a mammalian anima is gotten and frozen. Then, the epidermis is removed by decellularization to obtain a mammalian dermis without the epithelial cell nest. Next, the dermis is frozen and dried. Finally, the dermis is flattened by a flattening element to obtain the artificial skin.

In another prior art, the artificial skin having an epidermis layer and a dermal layer is obtained by combined polymeric material, such as a hydrogel.

However, the artificial sweat glands and hair follicles of the skin cannot be obtained either by the method of the mammal or the method of the polymeric material, so that the artificial skin loses the functions of perspiration and hair follicles protection.

Thus, the above-mentioned conventional technology still has a lot of missing, and it is not a good designer. It needs to be improved. In view of the above, the present invention discloses a method for 3D printing artificial skin.

### SUMMARY OF THE INVENTION

An object of the present invention is to provide a method for 3D printing artificial skin. In an embodiment of the present invention, the method for 3D printing artificial skin of the present invention comprises the following steps of: S1: printing a dermis layer by a hydrogel combined with an autologous dermal cell; S2: printing an epidermis layer on the dermis layer by a hydrogel combined with an autologous kerationocytes cell; and S3: implanting a plurality of progenitor cells for sweat glands and hair follicles through the epidermis layer into the dermis layer to form an artificial skin.

Wherein, the autologous dermal cell, the autologous keratinocytes cell and the progenitor cells for sweat glands and hair follicles are obtained by using an autologous skin separation process.

In addition, the method of 3D printing artificial skin of the present invention further comprises the following steps: S4: culturing the artificial skin; wherein step S4 is performed by a 3D culture bioreactor for accelerating a formation of the artificial skin structure and a growth of the progenitor cells for sweat glands and hair follicles.

In the practical application, steps S1 to S4 of the present invention are performed with a 3D printer, and a gas with a pressure higher than an atmospheric pressure is inputted into the 3D printer.

The steps S1 to S4 can be completed by the 3D printer so that the artificial skin is not contaminated by the outer container during the manufacturing stage. The 3D printer works in the positive pressure environment, so that protection of the production environment is not contaminated by external bacteria to ensure that the artificial skin will not be infected by bacteria before use.

Compared to the prior art, the method for 3D printing artificial skin of the present invention print the dermis layer and the epidermis layer by the separation cell from the autologous skin, and implant the progenitor cells for sweat glands and hair follicles by the autologous skin separation cells into the dermis layer to form an artificial skin. Because all used materials are from the autologous skin, and the 3D printing technology is used to implant the progenitor cells for sweat glands and hair follicles to form the artificial skin which has sweat glands and hair follicle, the artificial skin obtained by the method for 3D printing artificial skin of the present invention is a complete skin structure which can be mass-produced, can be customized to adjust for sweat glands and hair follicles densities, and is sterile and highly biocompatible.

The advantages and spirit of the invention may be further understood by the following detailed description of the invention and the appended drawings.

### BRIEF DESCRIPTION OF THE APPENDED DRAWINGS

FIG. 1 shows a step flow chart of the method for 3D printing artificial skin in an embodiment of the present invention.

FIG. 2 shows a schematic diagram of the dermis layer of the method for 3D printing artificial skin in an embodiment of the present invention.

FIG. 3 shows a schematic diagram of the dermis layer and the epidermis layer of the method for 3D printing artificial skin in an embodiment of the present invention.

FIG. 4 shows a schematic diagram of the artificial skin containing the precursor cells for sweat glands and hair follicles of the method for 3D printing artificial skin in an embodiment of the present invention.

FIG. 5 shows a schematic diagram of the artificial skin after the culture of the method for 3D printing artificial skin in an embodiment of the present invention.

### DETAILED DESCRIPTION OF THE INVENTION

A detailed description of the hereinafter described embodiments of the disclosed apparatus and method are presented herein by way of exemplification and not limitation with reference to the Figures. Although certain embodiments are shown and described in detail, it should be understood that various changes and modifications can be made without departing from the scope of the appended claims. The scope of the present invention will in no way be limited to the number of constituting components, the materials thereof, the shapes thereof, the relative arrangement thereof, etc., and are disclosed simply as an example of embodiments of the present invention.

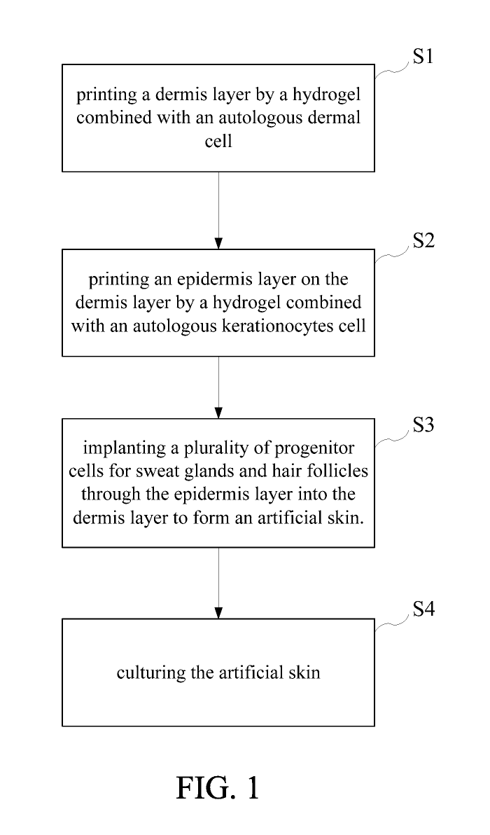

Please refer to FIG. 1. FIG. 1 shows a step flow chart of the method for 3D printing artificial skin in an embodiment of the present invention. In an embodiment of the present invention, the method for 3D printing artificial skin of the present invention comprises the following steps of: S1: printing a dermis layer 14 by a hydrogel 21 combined with an autologous dermal cell; S2: printing an epidermis layer 16 on the dermis layer 14 by a hydrogel 22 combined with an autologous kerationocytes cell; and S3: implanting a plurality of progenitor cells 23 for sweat glands and hair follicles through the epidermis layer 16 into the dermis layer 14 to form an artificial skin.

Wherein, the autologous dermal cell, the autologous keratinocytes cell and the progenitor cells 23 for sweat glands and hair follicles are obtained by using an autologous skin separation process.

In the practical application, the autologous dermal cell, the autologous keratinocytes cell and the progenitor cells 23 these are separated that can be proliferated by a culture procedure.

In the practical application, the hydrogel can be Chitosan, Gelatin, Alginate, Fibrinogen, Hyaluronic acid, Collagen or Glycerol.

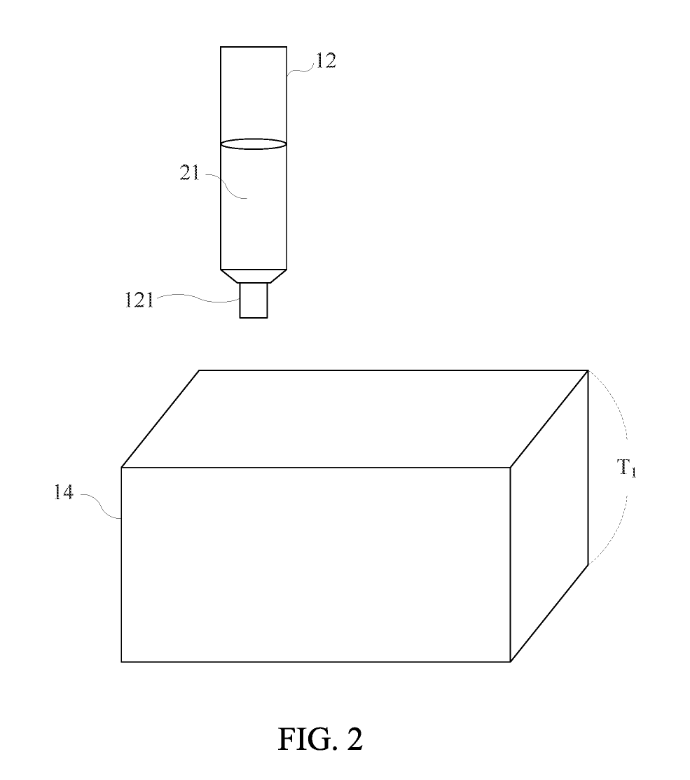

Please refer to FIG. 2. FIG. 2 shows a schematic diagram of the dermis layer 14 of the method for 3D printing artificial skin in an embodiment of the present invention. Step S1 prints the dermal layer 14 required for the autologous dermal cell and the hydrogel 21 in the print heads 121 of the 3D printer 12. Wherein, the thickness T₁ of the dermal layer 14 is 0.3 mm to 3 mm.

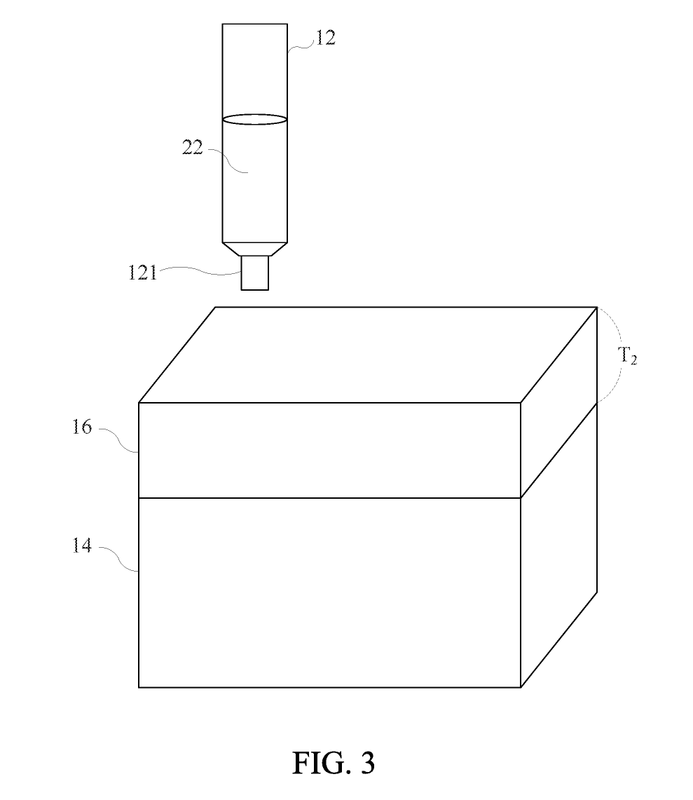

Please refer to FIG. 3. FIG. 3 shows a schematic diagram of the dermis layer 14 and the epidermis layer 16 of the method for 3D printing artificial skin in an embodiment of the present invention. Step S2 prints the epidermal layer 16 required for the autologous keratinocytes cell and the hydrogel 22 on the dermal layer 14 in the print heads 121 of the 3D printer 12. Wherein, the thickness T2 of the epidermal layer 16 is 0.03 mm to 0.3 mm.

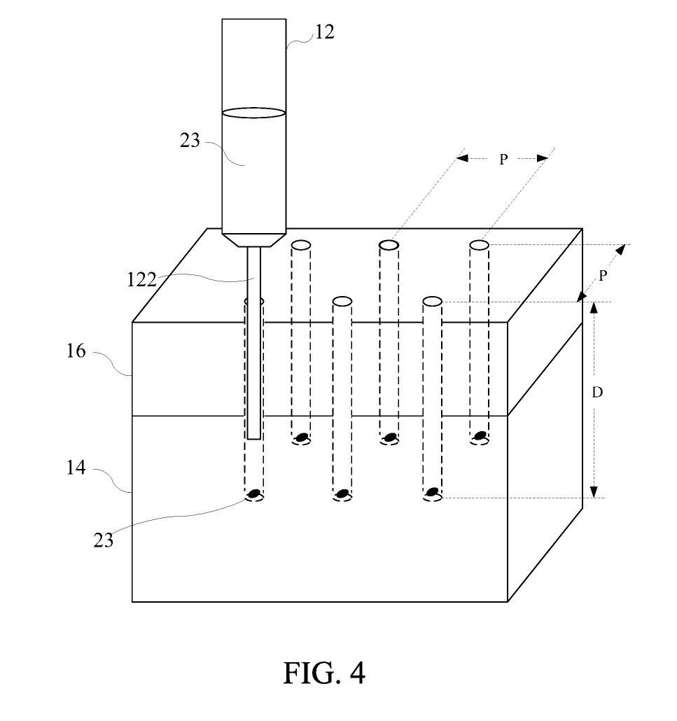

Please refer to FIG. 4. FIG. 4 shows a schematic diagram of the artificial skin containing the precursor cells 23 for sweat glands and hair follicles of the method for 3D printing artificial skin in an embodiment of the present invention. Step S3 is performed by using another print head 122 of a 3D printer 12 to puncture the epidermis layer 16 into the dermis layer 14 within a predetermined implanting pitch P so as to implant the progenitor cells 23 for sweat glands and hair follicles after reaching an implanting depth D.

Wherein, the predetermined implanting pitch P is 0.01 mm to 0.1 mm.

Furthermore, the implanting depth D is 0.2 mm to 2 mm.

In the practical application, the 3D printer 12 can control the pitch P to obtain a uniform sweat glands or hair follicles by precise positioning control or use different pitches P to obtain sweat glands and hair follicles of different densities. Therefore, the artificial skin containing the sweat glands and hair follicles can be customized to produce with different densities and depths.

The temperature used in step S3 can be 37° C., the pressure can be 0.1 to 0.2 bar, the tube diameter used can be 100 to 500 μm, and the valve opening time can be 100 to 700 μs. In addition, if the implantation of the sweat glands and hair follicle precursor cells 23 is successfully implanted, when injected into the dermal papilla cells, there will be sweat gland formation.

In the practical application, the implanting the progenitor cells 23 for sweat glands and hair follicles can include the following pretreatment methods: detection of cell activity, identification of cells, culture of cells, and amplification of cells.

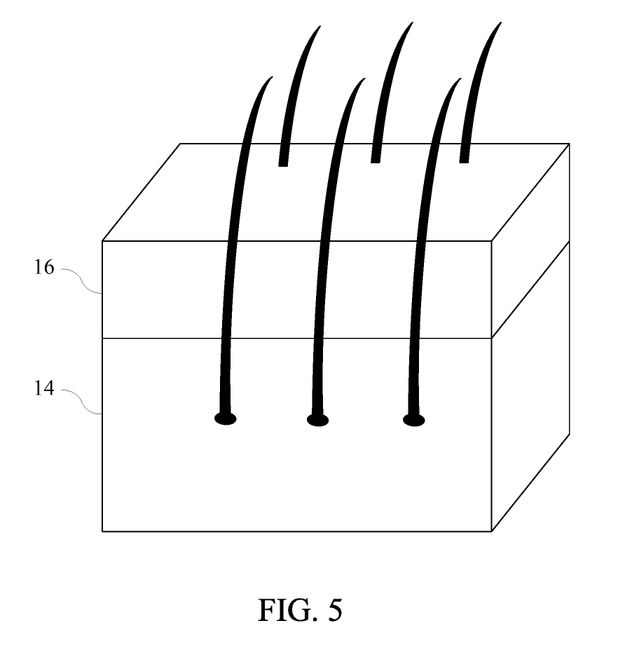

Please refer to FIG. 5. FIG. 5 shows a schematic diagram of the artificial skin after the culture of the method for 3D printing artificial skin in an embodiment of the present invention. In addition, the method of 3D printing an artificial skin of the present invention further comprises the following step: S4: culturing the artificial skin.

Wherein, step S4 is performed by a 3D culture bioreactor for accelerating a formation of the artificial skin structure and a growth of the progenitor cells 23 for sweat glands and hair follicles.

In the practical application, the artificial skin is placed in a Petri dish of a 3D culture bioreactor and injecting an appropriate amount of the culture fluid and gas (e.g. carbon dioxide). The artificial skin of the 3D culture bioreactor maintains an appropriate temperature and pressure, and rotating in the Petri dish centrifugal through the speed of 5 RPM˜30 RPM, to promote cells growth.

In addition, during the culture period, the culture fluid can be changed periodically, as well as gas and concentration. After a few days of culture, significant hair formation was observed. At this time, the cultured artificial skin had the function of perspiration and hair protection.

In the practical application, the steps S1 to S4 described in the method of 3D printing an artificial skin of present invention are performed with a 3D printer 12, and a gas with a pressure higher than an atmospheric pressure is inputted into the 3D printer 12. Preferably, the pressure of the print head is 0.2 psi and the pore size of the print head is 500 μm to increase cell viability.

The steps S1 to S4 can be completed by the 3D printer 12 so that the artificial skin is not contaminated by the outer container during the manufacturing stage. The 3D printer 12 works in the positive pressure environment, so that protection of the production environment is not contaminated by external bacteria to ensure that the artificial skin will not be infected by bacteria before use.

Further, the 3D printer 12 can input a gas, for helping the growth of the progenitor cells 23 for sweat glands and hair follicles, to enhance the yield of artificial skin.

The artificial skin produced by the present invention can be mass customized according to the skin shape. The artificial skin produced by the invention can effectively solve the problem of the secondary damage caused by the transplantation of autologous healthy skin or the biocompatibility problem of the external artificial skin for the patients with tertiary burns requiring skin grafting. The artificial skin produced by the present invention has sweat glands and hair follicles, so that can accelerate the wound recovery and restore the function of the original skin.

Compared to the prior art, the method for 3D printing artificial skin of the present invention print the dermis layer and the epidermis layer by the separation cell from the autologous skin, and implant the progenitor cells for sweat glands and hair follicles by the autologous skin separation cells into the dermis layer to form an artificial skin. Because all used materials are from the autologous skin, and the 3D printing technology is used to implant the progenitor cells for sweat glands and hair follicles to form the artificial skin which has sweat glands and hair follicles, the artificial skin obtained by the method for 3D printing artificial skin of the present invention is a complete skin structure which can be mass-produced, can be customized to adjust for sweat glands and hair follicles densities, and is sterile and highly biocompatible.

With the examples and explanations mentioned above, the features and spirits of the invention are hopefully well described. More importantly, the present invention is not limited to the embodiment described herein. Those skilled in the art will readily observe that numerous modifications and alterations of the device can be made while retaining the teachings of the invention. Accordingly, the above disclosure should be construed as limited only by the metes and bounds of the appended claims.

What is claimed is:

1. A method of 3D printing an artificial skin, comprising the following steps of:
S1: printing a dermis layer by a hydrogel combined with an autologous dermal cell;
S2: printing an epidermis layer on the dermis layer by a hydrogel combined with an autologous kerationocytes cell; and
S3: implanting a plurality of progenitor cells for sweat glands and hair follicles through the epidermis layer into the dermis layer to form an artificial skin.

2. The method for 3D printing the artificial skin of claim 1, wherein the autologous dermal cell, the autologous keratinocytes cell and the progenitor cells for sweat glands and hair follicles are obtained by using an autologous skin separation process.

3. The method for 3D printing artificial skin of claim 1, wherein the hydrogel is Chitosan, Gelatin, Alginate, Fibrinogen, Hyaluronic acid, Collagen or Glycerol.

4. The method for 3D printing the artificial skin of claim 1, wherein step S3 is performed by a 3D printer for implanting a pin to puncture the epidermis layer into the dermis layer within a predetermined implanting pitch so as to implant the progenitor cells for sweat glands and hair follicles after reaching an implanting depth.

5. The method for 3D printing the artificial skin of claim 4, wherein the implanting depth is 0.2 mm to 2 mm.

6. The method for 3D printing the artificial skin of claim 4, wherein the predetermined implanting pitch is 0.01 mm to 0.1 mm.

7. The method for 3D printing the artificial skin of claim 1, wherein a thickness of the dermal layer is 0.3 mm to 3 mm.

8. The method for 3D printing the artificial skin of claim 1, wherein a thickness of the epidermis layer is 0.03 mm to 0.3 mm.

9. The method for 3D printing the artificial skin of claim 1, further comprising the following steps of:
S4: culturing the artificial skin;
wherein step S4 is performed by a 3D culture bioreactor for accelerating a formation of the artificial skin structure and a growth of the progenitor cells for sweat glands and hair follicles.

10. The method for 3D printing the artificial skin of claim 9, wherein steps S1 to S4 are performed with a 3D printer, and a gas with a pressure higher than an atmospheric pressure is inputted into the 3D printer.
